(19) United States
(12) Patent Application Publication
(10) Pub. No.: US 2007/0260318 A1
(43) Pub. Date: Nov. 8, 2007

(54) Modular disc for spinal arthroplasty through a small posterior exposure with intradiscalor intervertebral assembly in-situ

(76) Inventor: Kevin Jon Lawson, Sault Ste. Marie, MI (US)

Correspondence Address: Robert Charles Hill, 235 Montgomery Street #821, San Francisco, CA, 94104, US

(21) Appl. No.: 11/416,905

(22) Filed: May 3, 2006

(51) Int. Cl. A61F 2/44

(52) U.S. Cl. 623/17.16

(57) ABSTRACT

A prosthetic nucleus replacement comprises modules about 10-13 mm in width of various heights that can be placed via a posterior approach and assembled intradiscalor intervertebral

### BACKGROUND OF THE INVENTION

1. Field of the Invention

The present invention relates to surgical methods and devices for spinal reconstruction, and in particular to the posterior surgical insertion of prosthetic nucleus replacement that is assembled from modules in-situ within the annulus fibrosis.

2. Description of Related Art

The spine is comprised of seven cervical, twelve thoracic, and five lumbar vertebrae, followed by fused sacral and coccygeal segments. The spine protects the spinal cord, bears loads during activity, and provides for motion. Vertebral bodies consist of an outer shell of cortical bone containing a cylindrical mass of cancellous bone.

The discs are fibrocartilagenous structures positioned between the vertebrae with a central core. Such core is the nucleus pulposus, a thick, gelatinous substance comprised of water and protein molecules. The nucleus pulposus is surrounded by multiple layers of fibers that comprise the ligamentous outer layer, or the annulus fibrosus. The annulus connects the discs to the vertebral bodies, and helps cushion the spine and control motion. Facet joints bilaterally allow articulation between the vertebrae and provide stability for the spine. The spine is surrounded anteriorly and posteriorly by many ligaments that provide support and constrain motion within safe anatomic limits.

The spine, like any other area of the skeleton, is susceptible to degenerative, age-related changes. The discs can gradually lose hydration as the protein molecules in the nucleus lose their capacity to bind water, causing the discs to become thinner, more fibrotic, and compressed with age. This in turn, can cause the annulus to bulge posteriorly into the canal. Aging discs can permit abnormal motion between the vertebrae. Bone spurs can develop from the vertebral bodies as a result of abnormal stresses and motion, narrowing the canal or neural foramen. Facet joints are diarthrodial joints, and are subject to age related changes, such as joint enlargement, spurs, and cysts. These changes can contribute to narrowing of the canal or the neural foramen. The ligamentum flavum and the posterior longitudinal ligament can thicken and become redundant as discs decrease in height. The bulging of these ligaments can occupy space in the central canal.

All these degenerative changes can contribute to stenosis, a narrowing of the space available for the spinal cord or nerve roots. Some individuals are predisposed to developing stenosis because of short pedicles that result in a congenitally small central spinal canal. Symptoms of lumbar stenosis are lower extremity pain, weakness/fatigue, and sensory changes. Symptoms of lumbar spinal stenosis are exacerbated by standing or walking. The pain of spinal stenosis is referred to as neurogenic claudication.

Trauma or cumulative wear and tear from age can result in disc herniations, a condition in which the annulus ruptures, allowing disc material to escape, most often posteriorly or posterolaterally. Such disc material can occupy space in the canal or neural foramen, potentially compromising the cord or nerve root at that level. If the nerve root is compressed or irritated by the disc material, radiculopathy may result. Radiculopathy characteristically causes pain along the nerve root distribution, sensory disturbance, and/or weakness in a myotomal distribution.

When the surgical approach for this type of procedure is from the back it is called a posterior lumbar interbody fusion (PLIF). PLIF surgery involves adding bone graft to an area of the spine to set up a biological response that causes the bone to grow between the two vertebral elements and thereby stop the motion at that segment. Unlike the posterolateral gutter fusion, PLIF achieves spinal fusion in the low back by inserting a bone graft and/or spinal implant directly into the disc space. A PLIF fusion is often supplemented by a simultaneous posterolateral spine fusion surgery.

In posterior lumbar interbody fusion surgery, the spine is approached through a three-inch to six-inch long incision in the midline of the back. The left and right lower back muscles are stripped off the lamina on both sides and at multiple levels. After the spine is approached, the lamina is removed and that allows visualization of the nerve roots. The facet joints, which are directly over the nerve roots, may then be undercut to give the nerve roots more room. The nerve roots are retracted to one side and the disc space is cleaned of the disc material. A bone graft, or anterior interbody cages with bone, is then inserted into the disc space and the bone grows from vertebral body to vertebral body.

A pure PLIF spine surgery can provide anterior fusion of the disc space without having a second incision as would be necessary with an anterior/posterior spine fusion surgery. But, not as much of the disc space can be removed with a posterior approach. An anterior approach provides for a much more comprehensive evacuation of the disc space and this leads to increase surface area available for a fusion. A much larger bone graft and/or spinal implant can be inserted from an anterior approach.

### SUMMARY OF THE INVENTION

An object of the present invention is to provide a prosthetic nucleus replacement that can be surgically implanted from the posterior below the spinal cord level.

Another object of the present invention is to provide a relatively large prosthetic nucleus replacement that can be implanted through a relatively small posterior exposure.

A further object of the present invention is to provide a prosthetic nucleus replacement that can achieve a disc height improvement or restoration, and retain valuable anatomic motions.

Briefly, a prosthetic nucleus replacement embodiment of the present invention comprises modules about 10-13 mm in width of various heights that can be placed via a posterior approach and assembled intradiscalor intervertebral.

An advantage of the present invention is that a prosthetic nucleus replacement is provided that flexibly supports the normal compressive loads experienced by natural vertebrae.

Another advantage of the present invention is that a prosthetic nucleus replacement is provided that can be implanted through a small posterior exposure.

The above and still further objects, features, and advantages of the present invention will become apparent upon consideration of the following detailed description of specific embodiments thereof, especially when taken in conjunction with the accompanying drawings.

### BRIEF DESCRIPTION OF THE DRAWINGS

FIG. 1 is a perspective diagram representing the surgical procedure to implant a prosthetic nucleus replacement embodiment of the present invention in the spine of a patient; and

FIG. 2 is a cross-section diagram representing the surgical procedure of FIG. 1

### DETAILED DESCRIPTION OF THE INVENTION

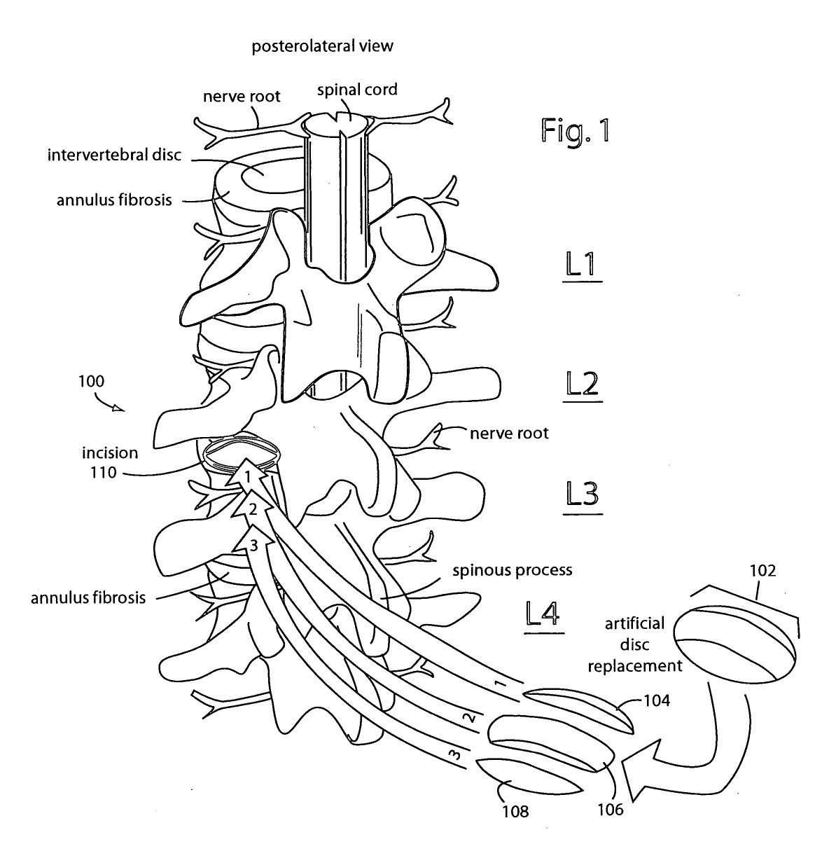

FIG. 1 represents a lower portion of the human spine 100. There are seven cervical (C1-C7), twelve thoracic (T1-T12), five lumbar (L1-L5), and five sacral (S1-S5) vertebrae. FIG. 1 illustrates the first four lumbar vertebrae, L1-L4. The thoracic vertebrae are defined by the spinal cord segments are not necessarily situated at the same vertebral levels. For example, while the C1 cord is located at the C1 vertebra, the C8 cord is situated at the C7 vertebra. While the T1 cord is situated at the T1 vertebra, the T12 cord is situated at the T8 vertebra. The lumbar cord is situated between T9 and T11 vertebrae. The sacral cord is situated between the T12 to L2 vertebrae.

The spinal roots for C1 exit the spinal column at the atlanto-occiput junction. The spinal roots for C2 exit the spinal column at the atlanto-axis. The C3 roots exit between C2 and C3. The C8 root exits between C7 and C8. The first thoracic root or T1 exits the spinal cord between T1 and T2 vertebral bodies. The T12 root exits the spinal cord between T1 and L1. The L1 root exits the spinal cord between L1 and L2 bodies. The L5 root exits the cord between L1 and S1 bodies.

The cervical cord innervates the deltoids (C4), biceps (C4-5), wrist extensors (C6), triceps (C7), wrist extensors (C8), and hand muscles (C8-T1). The thoracic vertebral segments are defined by those that have a rib. These vertebral segments form the back wall of the pulmonary cavity and the ribs. The spinal roots form the intercostal (between the ribs) nerves that run on the bottom side of the ribs and these nerves control the intercostal muscles and associated dermatomes. The lumbosacral vertebra form the remainder of the segments below the vertebrae of the thorax.

The lumbosacral spinal cord, however, starts at about T9 and continues only to L2. It contains most of the segments that innervate the hip and legs, as well as the buttocks and anal regions. The human spinal cord ends at L2 vertebral level. The tip of the spinal cord is called the conus. Below the conus, there is a spray of spinal roots that is frequently called the cauda equina or horse's tail. Injuries to T12 and L1 vertebra damage the lumbar cord. Injuries to L2 frequently damage the conus. Injuries below L2 usually involve the cauda equina and represent injuries to spinal roots rather than the spinal cord proper.

FIG. 1 illustrates a typical placement of a prosthetic spinal nucleus modular disc replacement embodiment of the present invention, referred to herein by the general reference numeral 102. The modular disc 102, in this instance, comprises three interlocking sections, 104, 106, and 108. As few as two such modular sections, and even four such sections can be used in embodiments of the present invention.

The point is to allow the in-situ assembly of a rather large disc 102 through a small posterior incision 110. In conventional procedures, the limitations of the allowable size of incision 110 have prevented the implantation of an adequately sized prosthesis from the posterior. An anterior approach, or an inadequate prosthesis, has been necessitated.

The modular parts 104, 106, and 108, are selected according to their heights and ability to fill the intervertebral spaces to restore normal anatomical function. They are typically 10-13 mm in width. Such selection can be done in real-time by the surgeon during the procedure. It may even be possible to engage in some trial-and-error to see which sizes of standard modules fit the intervertebral space the best.

The incision 110 is at the L2-3 level or below. At higher levels the presence of the spinal column interferes with the posterior surgical approach. The modular disc 102 could be implanted using the anterior method, albeit with a smaller incision than is conventional. In such case, disc 102 may be used at any level in the spine or neck.

Prosthetic nucleus replacement embodiments of the present invention may comprise a solid polymer flattened into an oval disk. In general, any solid biocompatible material can be used, including various polymers and plastics, titanium, stainless steel, tantalum, chrome cobalt alloys, etc. A number of different interlocking mechanisms and strategies can be employed, and these are design choices that need not be further detailed here. For example, adhesives, fasteners, dovetail joints, etc.

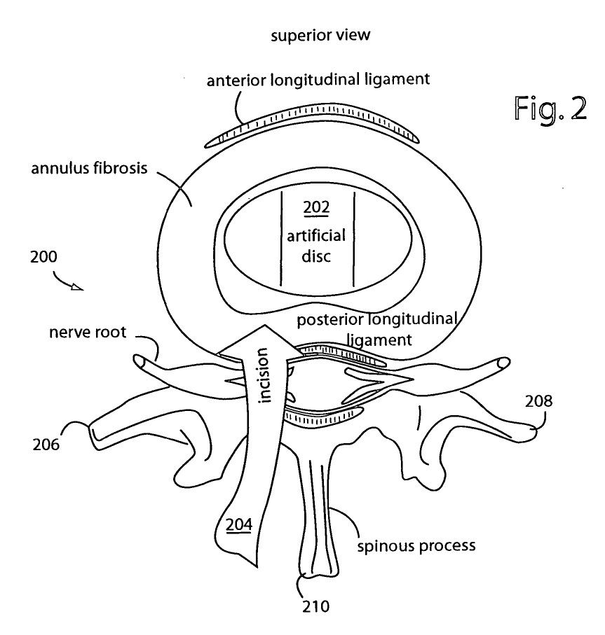

FIG. 2 illustrates the procedure of FIG. 1 in cross-section. An artificial disc 202 is piece-by-piece inserted posteriorly by a surgeon through an incision 204. Such incision here is shown over a left facet 206, but could also be approached over a right facet 208. The incision cannot be straight-in, due to spinous process 210. A retractor is used to pull the dura to one side during the procedure.

Although particular embodiments of the present invention have been described and illustrated, such was not intended to limit the invention. Modifications and changes will no doubt become apparent to those skilled in the art, and it was intended that the invention only be limited by the scope of the appended claims.

The invention is claimed, as follows:

1. A prosthetic nucleus disc replacement for implanting within an annulus fibrosis in a part of the human spine, comprising:
a first modular part of an ellipsoidal body having a convex top surface for contacting and articulating with an end-plate cartilage of a superior vertebrae and a bottom surface for contact with an inferior vertebrae; and
a second modular part of said ellipsoidal body able to lock onto the first modular part after both are serially inserted into an incision in a patient's spine;
wherein, the assembled modular parts restore anatomical function of said patients spine.

2. The prosthetic nucleus disc replacement of claim 1, wherein:
the first and second modular parts are selectable from a population of pieces with differing sizes to fit the intervertebral spaces following a discectomy.

3. The prosthetic nucleus disc replacement of claim 1, further comprising:
a third modular part of said ellipsoidal body able to lock onto the second modular part after the first and second are serially inserted into an incision in said patient's spine;
wherein, the first through third modular parts are selectable from a population of pieces with differing sizes to fit the intervertebral spaces following a discectomy.

4. The prosthetic nucleus disc replacement of claim 3, further comprising:
a fourth modular part of said ellipsoidal body able to lock onto the third modular part after the first through third are serially inserted into an incision in said patient's spine;
wherein, the first through fourth modular parts are selectable from a population of pieces with differing sizes to fit the intervertebral spaces following a discectomy.

5. A prosthetic nucleus replacement for implanting within an annulus fibrosis in one part of a human spine, comprising:
a first lateral section having an anterior edge and a posterior edge;
a second middle section able to lock into one side of the first lateral section when inserted from the posterior; and
a third lateral section able to lock into an opposite side of the second middle section when inserted from the posterior;
wherein, the first through third sections when interlocked with one another form an ellipsoidal body having a convex top surface for contacting and articulating with an end-plate cartilage of a superior vertebrae and a bottom surface for an immobile contact with an inferior vertebrae.

6. The prosthetic nucleus replacement of claim 5, wherein:
the first through third sections have heights and widths on the order of 10-13 millimeters, and said ellipsoidal body when assembled is approximately 20-30 mm long, anterior to posterior, and laterally about 30-39 mm wide.

7. A surgical method for implanting a prosthetic nucleus replacement within an annulus fibrosis in one part of a human spine, comprising:
making a posterolateral incision not larger than 15 mm in the back of a patient to one side of the spinous process to access the intervertebral space between a superior and an inferior vertebrae;
retracting the spinal column and/or nerve roots to provide access to said intervertebral space;
inserting into said incision a first lateral section with an anterior edge first, and pushing the whole once fully inserted into the intervertebral space to one side;
inserting into said incision a second middle section that locks into one side of said first lateral section when inserted from the posterior; and
inserting into said incision a third lateral section that locks into an opposite side of said second middle section when inserted from the posterior;
closing said incision;
wherein, the first through third sections when interlocked with one another in-situ form a prosthetic nucleus replacement larger than said incision.
